(19) United States
(12) Patent Application Publication
(10) Pub. No.: US 2003/0039636 A1
(43) Pub. Date: Feb. 27, 2003

(54) Novel self-inactivating (SIN) lentiviral vectors

(75) Inventors: Philippe Leboulch, Charlestown, MA (US); Karen Westerman, Reading, MA (US)

Correspondence Address: LAHIVE & COCKFIELD, 28 STATE STREET, BOSTON, MA, 02109, US

(73) Assignee: Genetix Pharmaceuticals, Inc., Cambridge, MA (US)

(21) Appl. No.: 10/137,788

(22) Filed: May 1, 2002

Related U.S. Application Data
(60) Provisional application No. 60/288,042, filed on May 1, 2001.

(51) Int. Cl. A61K 48/00; C12N 7/00; C12N 15/867

(52) U.S. Cl. 424/93.2; 435/456; 435/235.1

(57) ABSTRACT

Novel retroviral LTRs are disclosed for use in lentiviral gene therapy vectors. The R region of the LTR is derived from a lentivirus, but lacks all or a portion of the lentiviral TAR sequence to increase the safety of the gene therapy vector. The TAR sequence can be replaced by comparable sequences from the R region from a non-lentiviral retrovirus, thereby generating a hybrid lentiviral/non-lentiviral R region which lacks TAR. Also disclosed are gene therapy vectors including the LTRs and methods of using the vectors in lentiviral-based gene therapy.

### RELATED APPLICATIONS

This application claims priority to U.S. Provisional Application Serial No. 06/288,042, filed May 1, 2001, the entire contents of which are incorporated herein by this reference.

### BACKGROUND OF THE INVENTION

The success of gene therapy techniques depends largely on the ability to achieve a combination of stable chromosomal integration and high-level, regulated expression of transferred genes in a manner safe to humans. Many current techniques allow efficient transient transfection of cells in vitro and in vivo with large DNA fragments. However, subsequent chromosomal integration is very inefficient. To overcome low levels of integration, retroviral vectors, which integrate very efficiently in permissive cells, can be used.

While recombinant retroviral vectors allow for integration of a transgene into a host cell genome, most retroviruses can only transduce dividing cells, which limits their use for in vivo gene transfer to nonproliferating cells such as hepatocytes, myofibers, hematopoietic stem cells, and neurons. Non-dividing cells are the predominant, long-lived cell type in the body, and account for most desirable targets of gene transfer, including liver, muscle, and brain. Even protocols attempting the transduction of hematopoietic stem cells require demanding ex vivo procedures for triggering cell division in these cells prior to infection.

One way of overcoming this obstacle is to employ lentiviral vectors, in place of conventional retroviral vectors. Lentiviruses are complex retroviruses which, based on their higher level of complexity, can integrate into the genome of nonproliferating cells and modulate their life cycles, as in the course of latent infection. These viruses include HIV-1, HIV-2, SIV, FIV and EIV. Like other retroviruses, lentiviruses possess gag, pol and env genes which are flanked by two long terminal repeat (LTR) sequences. Each of these genes encodes multiple proteins, initially expressed as one precursor polyprotein. The gag gene encodes the internal structural (matrix capsid and nucleocapsid) proteins. The pol gene encodes the RNA-directed DNA polymerase (reverse transcriptase, integrase and protease). The env gene encodes viral envelope glycoproteins and additionally contains a cis-acting element (RRE) responsible for nuclear export of viral RNA.

The 5′ and 3′LTRs serve to promote transcription and polyadenylation of the virion RNAs and contains all other cis-acting sequences necessary for viral replication. Adjacent to the 5′LTR are sequences necessary for reverse transcription of the genome (the tRNA primer binding site) and for efficient encapsidation of viral RNA into particles (the Psi site). If the sequences necessary for encapsidation (or packaging of retroviral RNA into infectious virions) are missing from the viral genome, the result is a cis defect which prevents encapsidation of genomic RNA. However, the resulting mutant is still capable of directing the synthesis of all virion proteins. A comprehensive review of lentiviruses, such as HIV, is provided, for example, in Field's Virology (Raven Publishers), eds. B. N. Fields et al., ©1996.

Recently, a HIV-based lentiviral vector has been shown to be efficient in integrating into non-cycling cells (Verma, I. M. and N. Somia. 1997. Nature 389:239-30 242). However, it was found that the cellular expression of the lentivirus vector from the HIV LTR promoter/enhancer was poor (Choi, J. K. and A. Gewirtz. 1998. Blood 92:468a). FACS analysis of transduced hematopoietic cells demonstrated that fluorescent intensity in the lentiviral-transduced cells was only one-half log greater than control cells. Better expression was obtained by utilizing a HIV-based vector system that also expressed the viral transcription co-factor TAT that is critical for high expression off the HIV LTR. (Uchida et al. 1998. Proc. Natl. Acad. Sci. USA 95:11939-11944). Expression of TAT, however, compromises clinical safety because the TAT region is involved in viral replication.

Thus, in light of the danger of producing replication-competent HIV virus, safer HIV-based vectors capable of efficient expression are needed. In particular, self-inactivating lentivral vectors which cannot produce replication-competent viruses but are capable of efficiently infecting and integrating into the chromosomes of non-dividing cells, would be of immense value in human gene therapy.

### SUMMARY OF THE INVENTION

The present invention provides novel and improved self-inactivating (SIN) lentiviral vectors for use in gene therapy. The vectors are made safer by removing selected lentiviral sequences within the left (5′) and right (3′) LTRs to prevent replication-competant virus, while maintaining sequences necessary for obtaining high viral titers capable of infecting a wide variety of dividing as well as non-dividing cells.

In one embodiment of the invention, the lentiviral sequences removed from the LTRs are replaced with comparable sequences from a non-lentiviral retrovirus, thereby forming hybrid LTRs. In particular, the lentiviral R region within the LTR can be replaced in whole or in part by the R region from a non-lentiviral retrovirus. However, in all embodiments of the invention, the lentiviral TAR sequence, a sequence which interacts with TAT protein to enhance viral replication, is removed, preferably in whole, from the R region. The TAR sequence is then replaced with a comparable portion of the R region from a non-lentiviral retrovirus, thereby forming a hybrid R region. The LTRs can be further modified to remove and/or replace with non-lentiviral sequences all or a portion of the lentiviral U3 and U5 regions.

Accordingly, in one embodiment, the present invention provides a retroviral LTR comprising a hybrid lentiviral R region which lacks all or a portion of its TAR sequence, thereby eliminating any possible activation by TAT, wherein the TAR sequence or portion thereof is replaced by a comparable portion of the R region from a non-lentiviral retrovirus, thereby forming a hybrid R region. In a particular embodiment, the retroviral LTR comprises a hybrid R region, wherein the hybrid R region comprises a portion of the HIV R region (e.g., a portion comprising or consisting of the nucleotide sequence shown in SEQ ID NO: 10) lacking the TAR sequence, and a portion of the MoMSV R region (e.g., a portion comprising or consisting of the nucleotide sequence shown in SEQ ID NO: 9) comparable to the TAR sequence lacking from the HIV R region. In another particular embodiment, the entire hybrid R region comprises or consists of the nucleotide sequence shown in SEQ ID NO: 11.

Suitable lentiviruses from which the R region can be derived include, for example, HIV (HIV-1 and HIV-2), EIV, SIV and FIV. Suitable retroviruses from which non-lentiviral sequences can be derived include, for example, MoMSV, MoMLV, Friend, MSCV, RSV and Spumaviruse. In a particular embodiment exemplified herein, the lentivirus is HIV and the non-lentiviral retrovirus is MoMSV.

In another particular embodiment, the LTR comprising a hybrid R region is a left (5′) LTR and further comprises a promoter sequence upstream from the hybrid R region. Preferred promoters are non-lentiviral in origin and include, for example, the U3 region from a non-lentiviral retrovirus (e.g., the MoMSV U3 region). In a particular embodiment, the U3 region comprises the nucleotide sequence shown in SEQ ID NO: 12. In another embodiment, the left (5′) LTR further comprises a lentiviral U5 region downstream from the hybrid R region. In a preferred embodiment, the U5 region is the HIV U5 region including the HIV att site necessary for genomic integration. In another preferred embodiment, the U5 region comprises the nucleotide sequence shown in SEQ ID NO: 13. In yet another preferred embodiment, the entire left (5′) hybrid LTR comprises the nucleotide sequence shown in SEQ ID NO: 1.

In another particular embodiment, the LTR comprising a hybrid R region is a right (3′) LTR and further comprises a modified (e.g., truncated) lentiviral U3 region upstream from the hybrid R region. The modified lentiviral U3 region preferably includes the att sequence, but lacks any sequences having promoter activity, thereby causing the vector to be self-inactivating (SIN) in that viral transcription can not go beyond the first round of replication following chromosomal integration. In a particular embodiment, the modified lentiviral U3 region upstream from the hybrid R region consists of the 3′ end of a lentiviral (e.g., HIV) U3 region up to and including the lentiviral U3 att site. In a preferred embodiment, the U3 region comprises the nucleotide sequence shown in SEQ ID NO: 15. In another embodiment, the right (3′) LTR further comprises a polyadenylation sequence downstream from the hybrid R region. In another preferred embodiment, the polyadenylation sequence comprises the nucleotide sequence shown in SEQ ID NO: 16. In yet another preferred embodiment, the entire right (5′) LTR comprises the nucleotide sequence shown in SEQ ID NO: 2 or 17:

Accordingly, in yet another embodiment, the present invention provides a left (5′) retroviral LTR comprising three regions, U3, R and U5, wherein the U3 region comprises a promoter sequence, the R region comprises a combination of (i.e., is a hybrid of) a portion of an R region from a lentivirus which does not include the TAR sequence, and a portion of an R region from a non-lentiviral retrovirus comparable to the portion of the lentiviral R region containing TAR, and the U5 region comprises a lentiviral U5 region.

In a particular embodiment, the U3 region comprises the U3 region from a non-lentiviral retrovirus (e.g., the MoMSV U3 region), the R region comprises a hybrid of the HIV R region without all or a portion of the HIV TAR sequence but preferably including the HIV polyadenylation sequence (e.g., the right half of the HIV region), and a portion of the MoMSV R region which is comparable to the HIV TAR sequence missing from the HIV R region (e.g., the left half of the MoMSV R region), and the U5 region comprises the HIV U5 region including the HIV att sequence.

In yet another embodiment, the present invention provides a right (3′) retroviral LTR comprising three regions, U3, R and P, wherein the U3 region comprises the att sequence from a lentiviral U3 region but lacks any sequences from the U3 region having promoter activity, the R region comprises the same hybrid R region as in the left (5′) LTR of the invention described above, and the P region comprises a polyadenylation sequence. In a particular embodiment, the U3 region comprises the 3′ end of the HIV U3 region including the att sequence but lacking any U3 sequences upstream from the att sequence, the R region comprises a hybrid of a portion of the HIV R region lacking the TAR sequence, and a portion of the MoMSV R region comparable to TAR, and the P region comprises the rabbit β-globin gene polyadenylation sequence. In another embodiment, the right (3′) retroviral LTR further comprises the HIV central polypurine track (ppt) sequence upstream from the U3 region.

In yet a further embodiment, the present invention provides an expression vector (e.g., a lentiviral vector) comprising the above-described retroviral LTRs. The vector generally includes additional elements including, for example, all or a portion of a GAG sequence (e.g., including the packaging sequence), a central polypurine tract (cppt), one or more RNA export elements (e.g., the HIV Rev responsive element (RRE)), and one or more exogenous genes operably linked to a suitable promoter (e.g., the PGK, EF1 alpha and CMV promoter or the natural promoter associated with the gene) or internal ribosome entry site (IRES).

Vectors of the present invention can include any exogenous gene desired for delivery to a cell, including therapeutic genes for treating diseases. The vector can further include a marker gene (e.g., GFP) to help trace integration of the vector into the genome of the cell. In a particular embodiment, the gene encodes a protein which promotes angiogensis. Suitable genes include, but are not limited to genes encoding soluble Interleukin-1α Receptor Type I, Soluble Interleukin-1α Receptor Type II, Interleukin-1α Receptor Antagonist Protein (IRAP), Insulin-Like Growth Factor (IGF), Tissue Inhibitors of Matrix Metallo-Proteinases (TIMP)-1,-2,-3,-4, Bone Morphogenic Protein (BMP)-2 and -7, Indian Hedgehog, Sox-9, Interleukin-4, Transforming Growth Factor (TGF)-β, Superficial Zone Protein, Cartilage Growth and Differentiation Factors (CGDF), Bcl-2, Soluble Tumor Necrosis Factor (TNF)-α Receptor, Fibronectin and/or Fibronectin Fragments, Leukemia Inhibitory Factor (LIF), LIF binding protein (LBP), Interleukin-4, Interleukin-10, Interleukin-11, Interleukin-13, Hyaluronan Synthase, soluble TNF-α receptors 55 and 75, Insulin Growth Factor (IGF)-1, activators of plasminogen, urokinase plasminogen activator (uPA), parathyroid hormone-related protein (PTHrP), and platelet derived growth factor (PDGF)-AA -AB or -BB.

For delivery to cells, vectors of the present invention are preferably used in conjunction with a suitable packaging cell line or co-transfected into cells in vitro along with other vector plasmids containing the necessary retroviral genes (e.g., gag and pol) to form replication incompetent virions capable of packaging the vectors of the present invention and infecting cells.

Accordingly, in yet another embodiment, the invention provides a method of delivering a gene to a cell which is then integrated into the genome of the cell, comprising contacting the cell with a virion containing a vector of the present invention. The cell (e.g., in the form of tissue or an organ) can be contacted (e.g., infected) with the virion ex vivo and then delivered to a subject (e.g., a mammal, animal or human) in which the gene will be expressed. The cell can be autologous to the subject (i.e., from the subject) or it can be non-autologous (i.e., allogeneic or xenogenic) to the subject. Moreover, because the vectors of the present invention are capable of being delivered to both dividing and non-dividing cells, the cells can be from a wide variety including, for example, bone marrow cells, mesenchymal stem cells (e.g., obtained from adipose tissue), synovial fibroblasts, chondrocytes and other primary cells derived from human and animal sources. Alternatively, the virion can be directly administered in vivo to a subject or a localized area of a subject (e.g., localized vasculature).

Accordingly, the novel vectors of the present invention can be used a wide variety of gene therapy applications to provide a safe and effective method of achieving long-term gene expression.

### BRIEF DESCRIPTION OF THE FIGURES

FIG. 1 shows a complete map of the HIV-MoMSV hybrid vector, including (from left to right): 1) the hybrid 5′ LTR, contaning the MoMSV U3, the left half of the MoMSV R, the right half of the HIV R (without TAR) and the HIV U5 with att site; 2) the HIV packaging signal which is part of the sequence leading into the HIV GAG sequence; 3) the HIV GAG sequence, 4) the central polypurine tract (cppt), 5) the HIV RRE (rev response element); 6) the gene(s) to be expressed (e.g., GFP and/or a therapeutic gene of interest) coupled downstream from a suitable promoter (e.g., PGK driving the gene of interest) or IRES (internal ribosome entry site, which uses the PGK promoter driving a selection marker); 7) the polypurine tract leading into the 3′ LTR; and 8) the hybrid 3′LTR, including (from left to right) the HIV att sequence, the same hybrid MoMSV-HIV R as in the 5′ LTR and the rabbit β-globin polyadenylation sequence.

FIG. 2 shows a restriction map of the 5′ (left) hybrid LTR, including the cloning sites for the MoMSV 5′LTR U3, the left portion of the MoMSV 5′LTR R, the right portion of the HIV 5′ LTR R (no TAR), and the HIV 5′LTR U5 with att.

FIG. 3 shows a restriction map of the 3′ (right) hybrid LTR, including the cloning sites for the HIV E box and polypurine tract (ppt) linked to the HIV att sequence, the same hybrid MoMSV-HIV R as in the left LTR shown in FIG. 2, and the polyadenylation sequence.

FIG. 4A and 4B show the nucleotide sequence (SEQ ID NO: 1) of the complete hybrid 5′ (left) LTR as described in FIG. 2, including the start and end of the sequences corresponding to the MoMSV U3, the left portion of the MoMSV R, the right portion of the HIV R (no TAR), and the HIV U5 with att.

FIG. 5 shows the nucleotide sequence (SEQ ID NO: 2) of the complete hybrid 3′ (right) LTR as described in FIG. 3, including the start and end of the sequences corresponding to the HIV E box and polypurine tract (ppt) linked to the HIV att sequence, the same hybrid MoMSV-HIV R as in the left LTR, and the polyadenylation sequence.

FIG. 6 shows a restriction map of Plasmid A which contains the sequence at the start of the MoMSV 5′ LTR U3 region and was made by cloning annealed oligo (OPLB 10/11) into Puc 19.

FIG. 7 shows a restriction map of Plasmid B, containing the MoMSV 5′ LTR U3 region and part of the MoMSV R region, which was subcloned into Plasmid A.

FIG. 8 shows a restriction map of Plasmid C, containing the HIV 5′ LTR U5 region, the HIV R region with TAR deleted and the start of the HIV GAG sequence, which was subcloned into Puc 19. These HIV sequences were derived from plasmid HIVNL-43.

FIG. 9 shows a restriction map of Plasmid D which contains a combination of plasmids A, B, and C to form the 5′ hybrid MoMSV-HIV LTR. Plasmid D contains (within a Puc 19 backbone) the MoMSV U3 region with part the MoMSV R region, part of the HIV R region (lacking TAR), the HIV U5 region and the start of the HIV GAG sequence.

FIG. 10 shows a restriction map of Plasmid E containing the complete hybrid MoMSV-HIV 3′ LTR, including the HIV E box and ppt sequences, the att sequence of the HIV U3 region plus the following 37 nucleotides up to the start of the R region, the same hybrid R region as in the hybrid 5′ LTR and an ideal polyadenylation sequence.

### DETAILED DESCRIPTION OF THE INVENTION

Unlike previously described self-inactivating lentiviral vectors, the present invention provides a new type of self-inactivating (SIN) lentiviral vector in which the R region of the long terminal repeat (LTR) is replaced in whole or in part by the R region from a non-lentiviral retrovirus, thereby rendering the vector safer for use in gene therapy, while maintaing its ability to achieve high (therapeutically useful) viral titers in a variety of dividing and non-dividing cells.

In a particular embodiment of the invention exemplified herein, the lentiviral R region is replaced in part by the R region from a non-lentiviral retrovirus, thereby forming a hybrid lentiviral/non-lentiviral R region. More particularly, the R region comprises a hybrid between the R regions of Moloney Murine Leukemia Virus (MoMSV) and Human Immune Deficiency Virus 1 (HIV). The left portion of the R region corresponds to a portion of the MoMSV R region comparable to the portion of the HIV R region containing the tat reponsive (TAR) element. The term “comparable to” as used herein refers to sequences which achieve the same or similar viral titers. Thus, the left portion of the R region contains MoMSV sequences which, when replacing the portion of the HIV R region containing TAR, maintain the same or similar viral titers.

The right portion of the R region corresponds to a portion of the HIV R region including the poly A signal and structural components leading into the U5 region, but lacking the TAR element. In addition to this short segment of R, the only elements remaining from HIV within the LTRs of the vector are the U5 region, the packaging signal (GAG region), the polypurine tract and the right U3 att site, thereby significantly reducing HIV components in the vector.

The deletion of the TAR element eliminates any possible activation by tat, a region which interacts with the TAR element to enhance viral replication. Removal of the TAR element therefore eliminates the possibility of producing replication-competent HIV virus. Accordingly, the hybrid SIN vector of the present invention is safer for use in gene therapy than previously described SIN lentiviral vectors, while maintaining its ability to efficiently infect and integrate into non-dividing cells.

### DEFINITIONS

As used herein, the following terms and phrases used to describe the invention shall have the meanings provided below.

The term “retrovirus” refers to any known retrovirus (e.g., type c retroviruses, such as Moloney murine sarcoma virus (MoMSV), Harvey murine sarcoma virus (HaMuSV), murine mammary tumor virus (MuMTV), gibbon ape leukemia virus (GaLV), feline leukemia virus (FLV), spumavirus, Friend, Murine Stem Cell Virus (MSCV) and Rous Sarcoma Virus (RSV)). “Retroviruses” of the invention also include human T cell leukemia viruses, HTLV-1 and HTLV-2, and the lentiviral family of retroviruses, such as human Immunodeficiency viruses, HIV-1, HIV-2, simian immnodeficiency virus (SIV), feline immonodeficiency virus (FIV), equine immnodeficiency virus (EIV), and other classes of retroviruses.

“Retroviruses” are RNA viruses that utilize reverse transcriptase during their replication cycle. The retroviral genomic RNA is converted into double-stranded DNA by reverse transcriptase. This double-stranded DNA form of the virus is capable of being integrated into the chromosome of the infected cell; once integrated, it is referred to as a “provirus.” The provirus serves as a template for RNA polymerase II and directs the expression of RNA molecules which encode the structural proteins and enzymes needed to produce new viral particles. At each end of the provirus are structures called “long terminal repeats” or “LTRs.” The LTR contains numerous regulatory signals including transcriptional control elements, polyadenylation signals and sequences needed for replication and integration of the viral genome. The viral LTR is divided into three regions called U3, R and U5. The U3 region contains the enhancer and promoter elements. The U5 region is the sequence between the primer binding site and the R region and contains the polyadenylation sequence. The R (repeat) region is flanked by the U3 and U5 regions. The LTR composed of U3, R and U5 regions, appears at both the both the 5′ and 3′ ends of the viral genome.

The term “lentivirus” refers to a group (or genus) of retroviruses that give rise to slowly developing disease. Viruses included within this group include HIV (human immunodeficiency virus; including HIV type 1, and HIV type 2), the etiologic agent of the human acquired immunodeficiency syndrome (AIDS); visna-maedi, which causes encephalitis (visna) or pneumonia (maedi) in sheep, the caprine arthritis-encephalitis virus, which causes immune deficiency, arthritis, and encephalopathy in goats; equine infectious anemia virus, which causes autoimmune hemolytic anemia, and encephalopathy in horses; feline immunodeficiency virus (FIV), which causes immune deficiency in cats; bovine immune deficiency virus (BIV), which causes lymphadenopathy, lymphocytosis, and possibly central nervous system infection in cattle; and simian immunodeficiency virus (SIV), which cause immune deficiency and encephalopathy in sub-human primates. Diseases caused by these viruses are characterized by a long incubation period and protracted course. Usually, the viruses latently infect monocytes and macrophages, from which they spread to other cells. HIV, FIV, and SIV also readily infect T lymphocytes (i.e., T-cells).

The term “hybrid” refers to a vector, LTR or other nucleic acid containing both lentiviral sequences and non-lentiviral retroviral sequences.

The term “vector” refers to a nucleic acid molecule capable of transporting another nucleic acid to which it has been linked. The term “expression vector” includes any vector, (e.g., a plasmid, cosmid or phage chromosome) containing a gene construct in a form suitable for expression by a cell (e.g., linked to a promoter). In the present specification, “plasmid” and “vector” are used interchangeably, as a plasmid is a commonly used form of vector. Moreover, the invention is intended to include other vectors which serve equivalent functions.

The term “retroviral vector” refers to a vector containing structural and functional genetic elements that are primarily derived from a retrovirus.

The term “lentiviral vector” refers to a vector containing structural and functional genetic elements outside the LTRs that are primarily derived from a lentivirus.

The term “self-inactivating vector” refers to vectors in which the right (3′) LTR enhancer-promoter region, know as the U3 region, has been modified (e.g., by deletion or substitution) to prevent viral transcription beyond the first round of viral replication. Consequently, the vectors are capable of infecting and then integrating into the host genome only once, and can not be passed further. This is because the right (3′) LTR U3 region is used as a template for the left (5′) LTR U3 region during viral replication and, thus, the viral transcript can not be made without the U3 enhancer-promoter. If the viral transcript is not made, it can not be processed or packaged into virions, hence the life cycle of the virus ends. Accordingly, SIN vectors greatly reduce risk of creating unwanted replication-competent virus since the right (3′) LTR U3 region has been modified to prevent viral transcription beyond the first round of replication, hence eliminating the ability of the virus to be passed.

The term “long terminal repeat (LTR)” refers to domains of base pairs located at the ends of retroviral DNAs which, in their natural sequence context, are direct repeats and contain U3, R and U5 regions. LTRs generally provide functions fundamental to the expression of retroviral genes (e.g., promotion, initiation and polyadenylation of gene transcripts) and to viral replication.

The term “TAR” refers to the “trans-activation response” genetic element located in the R region of lentiviral (e.g., HIV) LTRs. This element interacts with the lentiviral trans-activator (tat) genetic element to enhance viral replication.

The term “R region” refers to the region within retroviral LTRs beginning at the start of the capping group (i.e., the start of transcription) and ending immediately prior to the start of the poly A tract. The R region is also defined as being flanked by the U3 and U5 regions. The R region plays an important role during reverse transcription in permitting the transfer of nascent DNA from one end of the genome to the other.

The term “transfection” refers to the introduction of foreign DNA into eukaryotic cells. Transfection may be accomplished by a variety of means known in the art including but not limited to calcium phosphate-DNA co-precipitation, DEAE-dextran-mediated transfection, polybrene-mediated transfection, electroporation, microinjection, liposome fusion, lipofection, protoplast fusion, retroviral infection, and biolistics.

The term “transduction” refers to the delivery of a gene(s) using a viral or retroviral vector by means of viral infection rather than by transfection. In preferred embodiments, retroviral vectors are transduced by packaging the vectors into virions prior to contact with a cell. For example, an anti-HIV gene carried by a retroviral vector can be transduced into a cell through infection and provirus integration.

The term “promoter/enhancer” refers to a segment of DNA which contains sequences capable of providing both promoter and enhancer functions. For example, the long terminal repeats of retroviruses contain both promoter and enhancer functions. The enhancer/promoter may be “endogenous” or “exogenous” or “heterologous.” An “endogenous” enhancer/promoter is one which is naturally linked with a given gene in the genome. An “exogenous” or “heterologous” enhancer/promoter is one which is placed in juxtaposition to a gene by means of genetic manipulation (i.e., molecular biological techniques) such that transcription of that gene is directed by the linked enhancer/promoter.

Efficient expression of recombinant DNA sequences in eukaryotic cells requires expression of signals directing the efficient termination and polyadenylation of the resulting transcript. Transcription termination signals are generally found downstream of the polyadenylation signal. The term “poly A site” or “poly A sequence” as used herein denotes a DNA sequence which directs both the termination and polyadenylation of the nascent RNA transcript. Efficient polyadenylation of the recombinant transcript is desirable as transcripts lacking a poly A tail are unstable and are rapidly degraded. The poly A signal utilized in an expression vector may be “heterologous” or “endogenous.” An endogenous poly A signal is one that is found naturally at the 3′ end of the coding region of a given gene in the genome. A heterologous poly A signal is one which is one which is isolated from one gene and placed 3′ of another gene.

The term “export element” refers to a cis-acting post-transcriptional regulatory element which regulates the transport of an RNA transcript from the nucleus to the cytoplasm of a cell. Examples of RNA export elements include, but are not limited to, the human immunodeficiency virus (HIV) rev response element (RRE) (see e.g., Cullen et al. (1991) J. Virol. 65: 1053; and Cullen et al. (1991) Cell 58: 423-426), and the hepatitis B virus post-transriptional regulatory element (PRE) (see e.g., Huang et al. (1995) Molec. and Cell. Biol. 15(7): 3864-3869; Huang et al. (1994) J. Virol. 68(5): 3193-3199; Huang et al. (1993) Molec. and Cell. Biol. 13(12): 7476-7486), and U.S. Pat. No. 5,744,326). Generally, the RNA export element is placed within the 3′ UTR of a gene, and can be inserted as one or multiple copies. RNA export elements can be inserted into any or all of the separate vectors generating the packaging cell lines of the present invention.

The phrase “retroviral packaging cell line” refers to a cell line (typically a mammalian cell line) which contains the necessary coding sequences to produce viral particles which lack the ability to package RNA and produce replication-competent helper-virus. When the packaging function is provided within the cell line (e.g., in trans by way of a plasmid vector), the packaging cell line produces recombinant retrovirus, thereby becoming a “retroviral producer cell line.”

### Self-Inactivating (SIN) Lentiviral Hybrid Vectors

Retroviral vectors of the present invention can be formed using standard cloning techniques by combining the desired DNA sequences in the order and orientation described herein (Current Protocols in Molecular Biology, Ausubel, F. M. et al. (eds.) Greene Publishing Associates, (1989), Sections 9.10-9.14 and other standard laboratory manuals; Eglitis, et al. (1985) Science 230:1395-1398; Danos and Mulligan (1988) Proc. Natl. Acad. Sci. USA 85:6460-6464; Wilson et al. (1988) Proc. Natl. Acad. Sci. USA 85:3014-3018; Armentano et al. (1990) Proc. Natl. Acad. Sci. USA 87:6141-6145; mammalian cell line) which contains the necessary coding sequences to produce viral particles which lack the ability to package RNA and produce replication-competent helper-virus. When the packaging function is provided within the cell line (e.g., in trans by way of a plasmid vector), the packaging cell line produces recombinant retrovirus, thereby becoming a “retroviral producer cell line.”

### Self-Inactivating (SIN) Lentiviral Hybrid Vectors

Retroviral vectors of the present invention can be formed using standard cloning techniques by combining the desired DNA sequences in the order and orientation described herein (Current Protocols in Molecular Biology, Ausubel, F. M. et al. (eds.) Greene Publishing Associates, (1989), Sections 9.10-9.14 and other standard laboratory manuals; Eglitis, et al. (1985) Science 230:1395-1398; Danos and Mulligan (1988) Proc. Natl. Acad. Sci. USA 85:6460-6464; Wilson et al. (1988) Proc. Natl. Acad. Sci. USA 85:3014-3018; Armentano et al. (1990) Proc. Natl. Acad. Sci. USA 87:6141-6145; Huber et al. (1991) Proc. Natl. Acad. Sci. USA 88:8039-8043; Ferry et al. (1991) Proc. Natl. Acad. Sci. USA 88:8377-8381; Chowdhury et al. (1991) Science 254:1802-1805; van Beusechem et al. (1992) Proc. Natl. Acad. Sci. USA 89:7640-7644; Kay et al. (1992) Human Gene Therapy 3:641-647; Dai et al. (1992) Proc. Natl. Acad. Sci. USA 89:10892-10895; Hwu et al. (1993) J. Immunol. 150:4104-4115; U.S. Pat. No. 4,868,116; U.S. Pat. No. 4,980,286; PCT Application WO 89/07136; PCT Application WO 89/02468; PCT Application WO 89/05345; and PCT Application WO 92/07573).

Suitable sources for obtaining retroviral (i.e., both lentiviral and non-lentiviral) sequences for use in forming the vectors include, for example, genomic RNA and cDNAs available from commercially available sources, including the Type Culture Collection (ATCC), Rockville, Md. The sequences also can be synthesized chemically.

For example, DNA sequences from retroviral LTRs can be cloned out of commercially purchased retroviral plasmids using PCR and other standard techniques as described in the Examples herein. Once the necessary sequences have been cloned, they can be combined in the desired order within a suitable expression vector, such as a pUC vector (e.g., pUC19) (University of California, San Francisco), pBR322, or pcDNA1 (InVitrogen) to form hybrid LTRs. The LTRs can then be modified by deletion (using e.g., restriction enzymes), substitution (using e.g., site directed mutagenesis), or addition so that they contain the desired U3, R, U5 and P sequences taught by the present invention. Once formed, the hybrid LTRs can then be cloned into a suitable lentiviral vector to form a hybrid vector.

In one embodiment of the invention, the portion of the HIV R region (within lentiviral vectors to infect both dividing and non-dividing cells and, more preferably, are capable of infecting substantially the same host range as fully lentiviral vectors.

Accordingly, to determine what portions of the lentiviral LTR U3, R and U5 regions can be deleted and not replaced at all (i.e., are non-essential) and which portions are essential to retain therapeutic value, various deletions can be made in these regions and the resulting vectors can be tested in such functional assays (e.g., which measure viral titers and/or host range) to assess whether therapeutic value has been maintained. For example, it was determined by way of the present invention that the entire left (5′) LTR U3 region of HIV can be deleted and replaced by a suitable promoter sequence, such as a non-lentiviral U3 region. It was also determined that the entire right (3′) LTR U5 region of HIV can be deleted and merely replaced by a polyadenylation sequence.

The same analysis also can be used to determine what portions of non-lentiviral LTR U3, R and U5 regions can be substituted for (i.e., are comparable to) portions of the lentiviral U3, R and U5 regions. In brief, various portions of non-lentiviral U3, R and U5 regions can be substituted into the corresponding lentiviral U3, R and U5 regions (in place of lentiviral sequences) and the resulting vectors can be tested to assess whether therapeutic value has been maintained. For example, it was determined by way of the present invention that the portion of the HIV R region containing the TAR element can be replaced by the corresponding left portion of the R region from a non-lentiviral retrovirus (e.g., MoMSV).

In particular embodiments, the present invention provides vectors containing hybrid LTRs comprising the particular nucleotide sequences shown in SEQ ID NOS: 1-17. The invention further includes nucleic acids and vectors containing nucleic acids which hybridize under high or low stringency conditions to nucleic acids having all or a portion of SEQ ID NOS: 1-17 and, in particular, SEQ ID NOS: 1, 2 and 9-17. Appropriate stringency conditions which promote DNA hybridization, for example, 6.0× sodium chloride/sodium citrate (SSC) at about 45° C., followed by a wash of 2.0× SSC at 50°, are known to those skilled in the art or can be found in Current Protocols in Molecular Biology, John Wiley & Sons, N.Y. (1989), 6.3.1-6.3.6. For example, the salt concentration in the wash step can be selected from a low stringency of about 2.0× SSC at 50° C. to a high stringency of about 0.2× SSC at 50° C. In addition, the temperature in the wash step can be increased from low stringency conditions at room temperature, about 22° C., to high stringency conditions at about 65° C. Preferred nucleic acids are at least 60-70% homologous, more preferably 80-90% homologous, and most preferably 95-98% homologous with the nucleic acid sequence of SEQ ID NOS: 1-17.

Thus, the invention encompasses nucleic acids which have a nucleotide sequence which is substantially similar to the nucleotide sequence shown in SEQ ID NOS: 1, 2 and 9-17 or encoding a protein having an amino acid sequence which is substantially similar to the amino acid sequences encoded by the nucleotide sequences shown in SEQ ID NOS: 1, 2 and 9-17. The term “substantially similar“, in regards to a nucleotide or amino acid sequence, means those nucleotide and amino acid sequences which have slight or inconsequential sequence variations from the sequences disclosed in SEQ ID NOS: 1, 2 and 9-17, e.g., the homologous nucleic acid functions in substantially the same manner to produce substantially the same polypeptide having substantially the same activity as the actual sequence. It is expected that substitutions or alterations can be made in the nucleotide or amino acid sequence without affecting function of the nucleic acid or protein encoded therein. For example, the degeneracy of the genetic code enables a number of amino acids to be designated by more than one triplet codon (for example, CAU and CAC both code for histidine). Thus, changes in the nucleotide sequence of SEQ ID NOS: 1, 2 and 9-17 (especially those within the third base of a codon) can be made which result in “silent” mutations in the DNA which do not affect the amino acid encoded. These silent mutations may occur naturally within a population (DNA polymorphism) or can be introduced by standard recombinant DNA techniques. Additionally, it should be appreciated by those skilled in the art that DNA sequence polymorphisms that do lead to changes in the amino acid sequences corresponding to the nucleotide sequences shown in SEQ ID NOS: 1, 2 and 9-17 may exist within a population due to natural allelic variation, or can be created by standard techniques, without changing the functional activity of the protein. Accordingly, nucleic acids having a nucleotide sequence substantially similar to SEQ ID NOS: 1, 2 and 9-17 or encoding a protein having an amino acid sequence substantially similar to the amino acid sequences corresponding to the nucleotide sequences shown in SEQ ID NOS: 1, 2 and 9-17 are intended to be encompassed by the invention.

A nucleic acid of the invention can be isolated by standard molecular biology techniques based upon the nucleotide sequences shown in SEQ ID NOS: 1, 2 and 9-17. For example, a labeled nucleic acid probe having a nucleotide sequence corresponding to all or part of SEQ ID NOS: 1-17 can be used to screen a cDNA or genomic DNA library. For instance, a nucleic acid of the invention can be isolated by selectively amplifying the nucleic acid using the polymerase chain reaction (PCR) method. For example, retroviral RNA can be isolated from infected cells (e.g., by the guanidinium-thiocyanate extraction procedure of Chirgwin et al. (1979) Biochemistry 18: 5294-5299) and cDNA can be prepared using reverse transcriptase.

A nucleic acid of the invention can be amplified from cDNA (or, alternatively, genomic DNA) using these oligonucleotide primers and standard PCR amplification techniques. The nucleic acid so amplified can be cloned into an appropriate vector and characterized by DNA sequence analysis.

Any suitable expression vector can be employed for generating vectors of the allelic variation, or can be created by standard techniques, without changing the functional activity of the protein. Accordingly, nucleic acids having a nucleotide sequence substantially similar to SEQ ID NOS: 1, 2 and 9-17 or encoding a protein having an amino acid sequence substantially similar to the amino acid sequences corresponding to the nucleotide sequences shown in SEQ ID NOS: 1, 2 and 9-17 are intended to be encompassed by the invention.

A nucleic acid of the invention can be isolated by standard molecular biology techniques based upon the nucleotide sequences shown in SEQ ID NOS: 1, 2 and 9-17. For example, a labeled nucleic acid probe having a nucleotide sequence corresponding to all or part of SEQ ID NOS: 1-17 can be used to screen a cDNA or genomic DNA library. For instance, a nucleic acid of the invention can be isolated by selectively amplifying the nucleic acid using the polymerase chain reaction (PCR) method. For example, retroviral RNA can be isolated from infected cells (e.g., by the guanidinium-thiocyanate extraction procedure of Chirgwin et al. (1979) Biochemistry 18: 5294-5299) and cDNA can be prepared using reverse transcriptase.

A nucleic acid of the invention can be amplified from cDNA (or, alternatively, genomic DNA) using these oligonucleotide primers and standard PCR amplification techniques. The nucleic acid so amplified can be cloned into an appropriate vector and characterized by DNA sequence analysis.

Any suitable expression vector can be employed for generating vectors of the present invention. As described in Examples below, suitable expression constructs include human cytomegalovirus (CMV) immediate early promoter constructs. The cytomegalovirus promoter can be obtained from any suitable source. For example, the complete cytomegalovirus enhancer-promoter can be derived from the human cytomegalovirus (hCMV). Other suitable sources for obtaining CMV promoters include commercial sources, such as Clontech, Invitrogen and Stratagene. Part or all of the CMV promoter can be used in the present invention. Other examples of constructs which can be used to practice the invention include constructs that use MuLV, SV40, Rous Sarcoma Virus (RSV), vaccinia P7.5, PGK, EF-1-alpha and rat β -actin promoters. In some cases, such as the RSV and MuLV, these promoter-enhancer elements are located within or adjacent to the LTR sequences.

Suitable regulatory sequences required for gene transcription, translation, processing and secretion are art-recognized, and are selected to direct expression of the desired protein in an appropriate cell. Accordingly, the term “regulatory sequence”, as used herein, includes any genetic element present 5′ (upstream) or 3′ (downstream) of the translated region of a gene and which control or affect expression of the gene, such as enhancer and promoter sequences. Such regulatory sequences are discussed, for example, in Goeddel, Gene expression Technology: Methods in eukaryotic promoters responsive to metal ions (e.g., the metallothionein promoter), glucocorticoid hormones and 2) the LacSwitch™ Inducible Mammalian Expression System (Stratagene) of E. coli. Briefly, in the E. coli lactose operon, the Lac repressor binds as a homotetramer to the lac operator, blocking transcription of the lac2 gene. Inducers such as allolactose (a physiologic inducer) or isopropyl-β-D-thiogalactoside (IPTG, a synthetic inducer) bind to the Lac repressor, causing a conformational change and effectively decreasing the affinity of the repressor for the operator. When the repressor is removed from the operator, transcription from the lactose operon resumes.

In yet another approach, selective expression of retroviral genes contained within the vectors of the invention can be achieved by cloning in a Cre/lox repressor system upstream of selected coding sequences. Specifically, a polystop signal can be inserted between the gene(s) to be selectively expressed and a 5′ promoter. The polystop signal is flanked by two loxP1 sites (Sauer (1993) Methods in Enzymology 225:890-900). Upon contact with cre recombinase, the lox sites will recombine and delete the polystop signal, allowing the promoter to act in cis to turn on expression of the gene(s).

### Packaging Cell Lines

Any suitable packaging system (cell line) can be employed with the hybrid vectors of the present invention to facilitate transduction of host cells with the vectors in gene therapy. Generally, the packaging cells are mammalian cells, such as human cells. Suitable human cell lines which can be used include, for example, 293 cells (Graham et al. (1977) J. Gen. Virol., 36:59-72, tsa 201 cells (Heinzel et al. (1988) J. Virol., 62:3738), and NIH3T3 cells (ATCC)). Other suitable packaging cell lines for use in the present invention include other human cell line derived (e.g., embryonic cell line derived) packaging cell lines and murine cell line derived packaging cell lines, such as Psi-2 cells (Mann et al. (1983) Cell, 33:153-159; FLY (Cossett et al. (1993) Virol., 193:385-395; BOSC 23 cells (Pear et al. (1993) PNAS 90:8392-8396; PA317 cells (Miller et al. (1986) Molec. and Cell. Biol., 6:2895-2902; Kat cell line (Finer et al. (1994) Blood, 83:43-50; GP+E cells and GP+EM12 cells (Markowitz et al. (1988) J. Virol., 62:1120-1124, and Psi Crip and Psi Cre cells (U.S. Pat. No. 5,449,614; Danos, O. and Mulligan et al. (1988) PNAS 85:6460-6464). Packaging cell lines of the present invention can produce retroviral particles having a pantropic amphotropic or ecotropic host range. Preferred packaging cell lines produce retroviral particles, such as lentiviral particles (e.g., HIV-1, HIV-2 and SIV) capable of infecting dividing, as well as non-dividing cells.

In a particular embodiment, the invention employs the split HIV gagpol packaging system, in which the HIV GAG-Protease (GAG-PR) sequence is on one plasmid and the HIV Reverse Transcriptase-integrase (RT-IN) sequence is fused to the VPR on a separate plasmid, described in U.S. Ser. No. 09/311,684 (U.S. Pat. No. 6,365,150), the entire contents of which are incorporated by reference herein. In a preferred embodiment, the invention employs a split HIV gagpol packaging system, in which the HIV GAG-protease (GAG-PR) is on one plasmid, a portion of the HIV Reverse Transcriptase-integrase (RT-IN) sequence is fused to a VPR or VPX on a separate plasmid, and the remaining portion of the RT-IN is fused to a VPR or VPX on a third plasmid.

The packaging cell line may also provide for the hybrid vector to affect the range of host cells capable of being infected by providing a particular envelope protein (e.g., a non-lentiviral envelope protein).

### Cell Transfection and Screening

Hybrid vectors of the present invention can be transfected or transduced into host cells and tested for infectivity using standard transfection/transduction techniques. Generally cells are incubated (i.e., cultured) with the vectors or virions containing the vectors in an appropriate medium under suitable transfection conditions, as is well known in the art.

Positive packaging cell transformants (i.e., cells which have taken up and integrated the retroviral vectors) can be screened for using a variety of selection markers which are well known in the art. For example, marker genes, such as green fluorescence protein (GFP), hygromycin resistance (Hyg), neomycin resistance (Neo) and β-galactosidase (β-gal) genes can be included in the vectors and assayed for using e.g., enzymatic activity or drug resistance assays. Alternatively, cells can be assayed for reverse transcriptase (RT) activity as described by Goff et al. (1981) J. Virol. 38:239 as a measure of viral protein production. Cells can also be measured for production of viral titers as is known in the art.

Similar assays can be used to test for the production of unwanted, replication-competent helper virus. For example, marker genes, such as those described above, can be included in the “producer” vector containing the viral packaging sequence (Ψ) and LTRs. Following transient transfection of packaging cells with the producer vector, packaging cells can be subcultured with other non-packaging cells. These non-packaging cells will be infected with recombinant, replication-deficient retroviral vectors of the invention carrying the marker gene. However, because these non-packaging cells do not contain the genes necessary to produce viral particles (e.g., TAR region), they should not, in turn, be able to infect other cells when subcultured with these other cells. If these other cells are positive for the presence of the marker gene when subcultured with the non-packaging cells, then unwanted, replication-competent virus has been produced.

Accordingly, to test for the production of unwanted helper-virus, hybrid lentiviral vectors of the invention can be subcultured with a first cell line (e.g., NIH3T3 cells) which, in turn, is subcultured with a second cell line which is tested for the presence of a marker gene or RT activity indicating the presence of replication-competent helper retrovirus. Marker genes can be assayed for using e.g., FACS, staining and enzymatic activity assays, as is well known in the art.

### Uses In Gene Therapy

Hybrid lentiviral vector of the present invention can be used to transfer selected genes into dividing as well as non-dividing cells including, but not limited to, cells of the skin, gastrointestinal tissue, cardiac tissue, and neuronal tissue. Techniques for transfer of selected genes into tissue or cells using viral vectors are well-established in the art. Genes for selection and transfer via viral vectors are also well known. One of skill can thus use these established techniques with the hybrid lentiviral vector of the present invention to efficiently transfer selected genes to cells and mammals. The characteristics of high expression and safety make the hybrid vector of the present invention a desirable vector for gene transfer in human therapy.

Suitable genes which can be delivered via the hybrid vectors of the invention include any therapeutic gene. For example, genes involved in promoting angiogenesis to treat ischemia can be delivered, such as genes encoding soluble Interleukin-1α Receptor Type I, Soluble Interleukin-1α Receptor Type II, Interleukin-1α Receptor Antagonist Protein (IRAP), Insulin-Like Growth Factor (IGF), Tissue Inhibitors of Matrix Metallo-Proteinases (TIMP)-1,-2,-3,-4, Bone Morphogenic Protein (BMP)-2 and -7, Indian Hedgehog, Sox-9, Interleukin-4, Transforming Growth Factor (TGF)-β, Superficial Zone Protein, Cartilage Growth and Differentiation Factors (CGDF), Bcl-2, Soluble Tumor Necrosis Factor (TNF)-α Receptor, Fibronectin and/or Fibronectin Fragments, Leukemia Inhibitory Factor (LIF), LIF binding protein (LBP), Interleukin-4, Interleukin-10, Interleukin-11, Interleukin-13, Hyaluronan Synthase, soluble TNF-α receptors 55 and 75, Insulin Growth Factor (IGF)-1, activators of plasminogen, urokinase plasminogen activator (uPA), parathyroid hormone-related protein (PTHrP), and platelet derived growth factor (PDGF)-AA -AB or -BB.

Cells can be transfected or transduced either in vivo or ex vivo and then returned to a subject (see e.g., U.S. Pat. No. 5,399,346). Thus, the cells can be autologous (e.g., a bone marrow cell, mesenchymal stem cell obtained from adipose tissue, a synovial fibroblast or a chondrocyte) or non-autologous (i.e., allogeneic or xenogenic), such as cells from a cell line or from primary cells derived from a human or animal source.

### Equivalents

Those skilled in the art will recognize, or be able to ascertain using no more than routine experimentation, many equivalents to the specific embodiments of the invention described herein. Such equivalents are intended to be encompassed by the following claims. The entire contents of all references, patents and published patent applications cited throughout this application are hereby incorporated by reference.

### EXAMPLES

### Example 1

### Construction of Hybrid HIV-MoMSV Vector

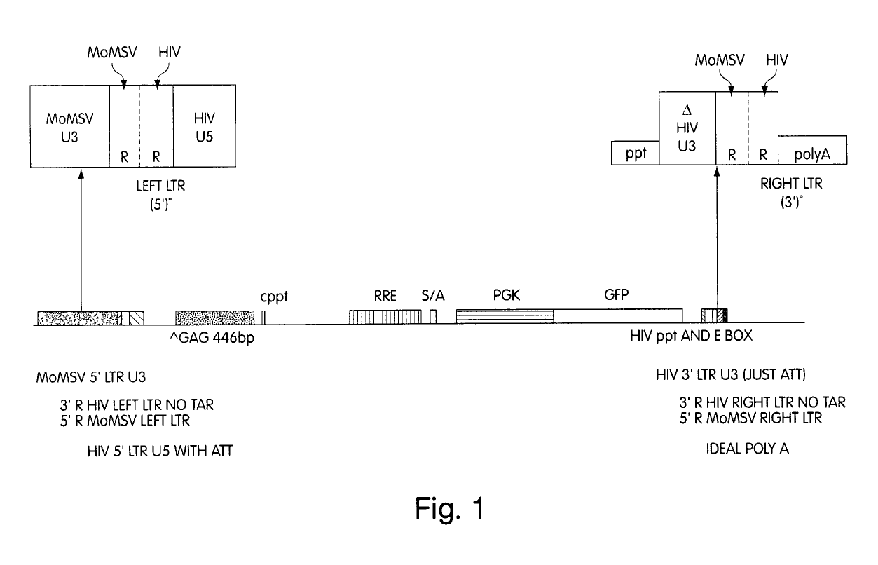

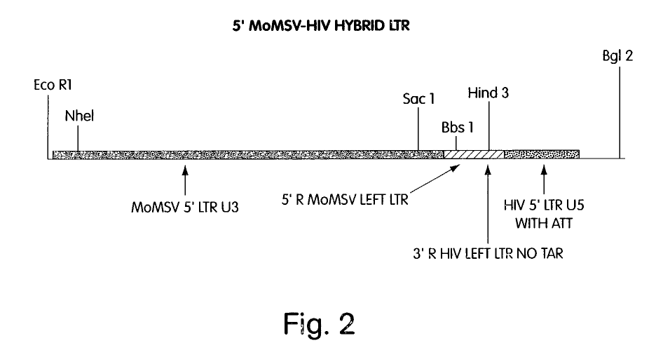

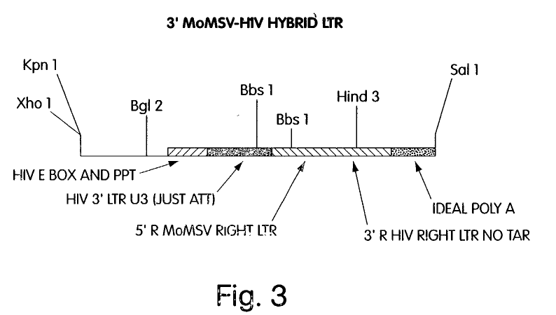

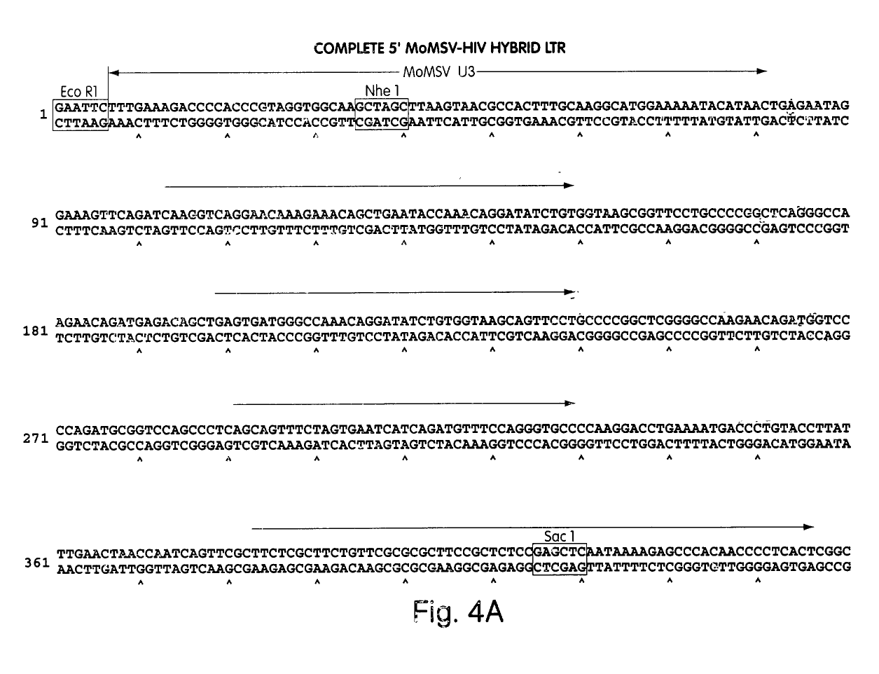

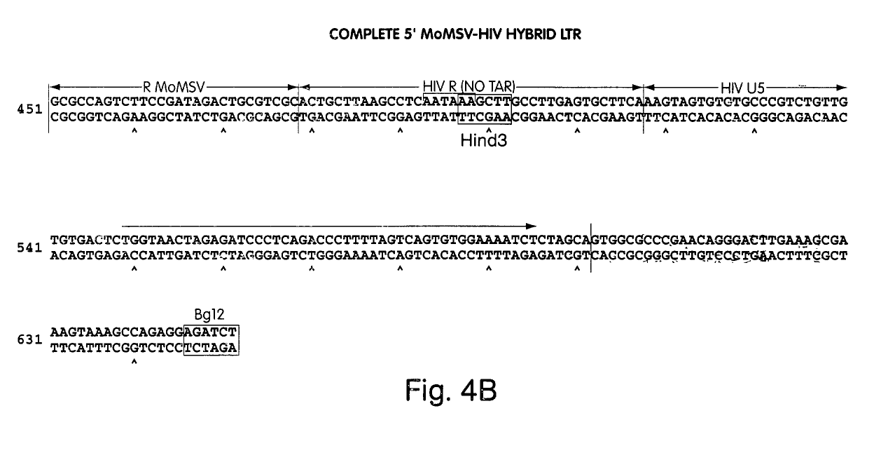

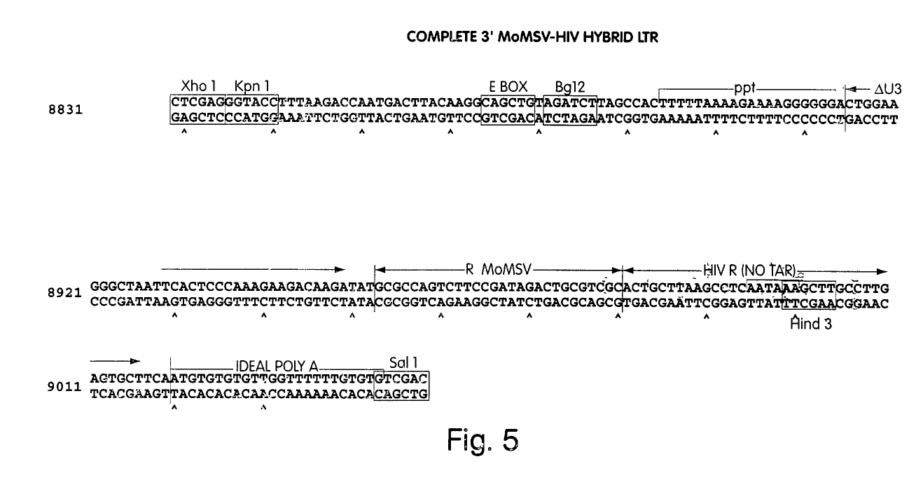

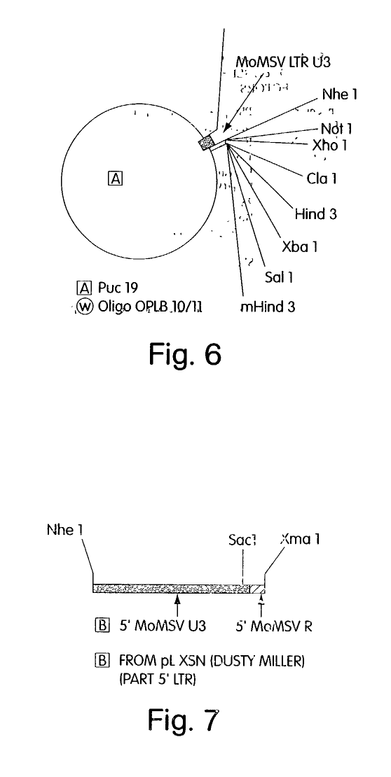

A hybrid retroviral vector in which portions of the U3, R and/or U5 regions from the HIV LTR were deleted or replaced by comparable portions of the LTR from MoMSV (as shown in FIG. 1) was constructed through a series of cloning steps combining pieces from several plasmids (as shown in FIGS. 6-10). Standard cloning techniques were used (e.g., Sambrook, J. et al. (1989) Molecular Cloning: A laboratory Manual-2nd. Cold Spring Harbor Laboratory Press, Cold Spring Harbor N.Y., USA).

The 5′ LTR (Plasmid D) was constructed by combining pieces from plasmids A, B and C.

The 3′ LTR (Plasmid E) was constructed by cloning a series of annealed oligonucleotides into Plasmid A.

The 5′ LTR along with the piece of gag from Plasmid D was cloned into Plasmid E creating a plasmid which contained both the 5′ and 3′ LTRs. Other components such as the RRE, selection marker and cppt were then subcloned into the plasmid containing both LTRs. The completed hybrid vector contained the following components: (from left to right): 1) a hybrid 5′ LTR, contaning the MoMSV U3, the left half of the MoMSV R, the right half of the HIV R (without TAR) and the HIV U5; 2) the HIV packaging signal which is part of the sequence leading into the HIV GAG sequence; 3) the HIV GAG sequence, 4) the HIV central polypurine tract (cppt), 5) the HIV RRE (rev response element); 6) the GFP gene coupled downstream from the PGK promoter; and 7) a polypurine tract leading into a hybrid 3′ LTR, including (from left to right) the HIV att sequence, the same hybrid MoMSV-HIV R as in the 5′ LTR and the rabbit β-globin polyadenylation sequence.

Plasmid A

Plasmid A (FIG. 6) was made by cloning annealed oligo OPLB 10/11 (SEQ ID NO: 3) into Puc 19. The OPLB 10/11 oligo contained the sequence for the start of the MoMSV 5′ LTR U3 region.

Plasmid B

Plasmid B (FIG. 7), a piece of plasmid PLXSN (Dusty Miller), was subcloned into Plasmid A, forming the MoMSV 5′ LTR U3 region and the left portion of the MoMSV R region.

Plasmid C

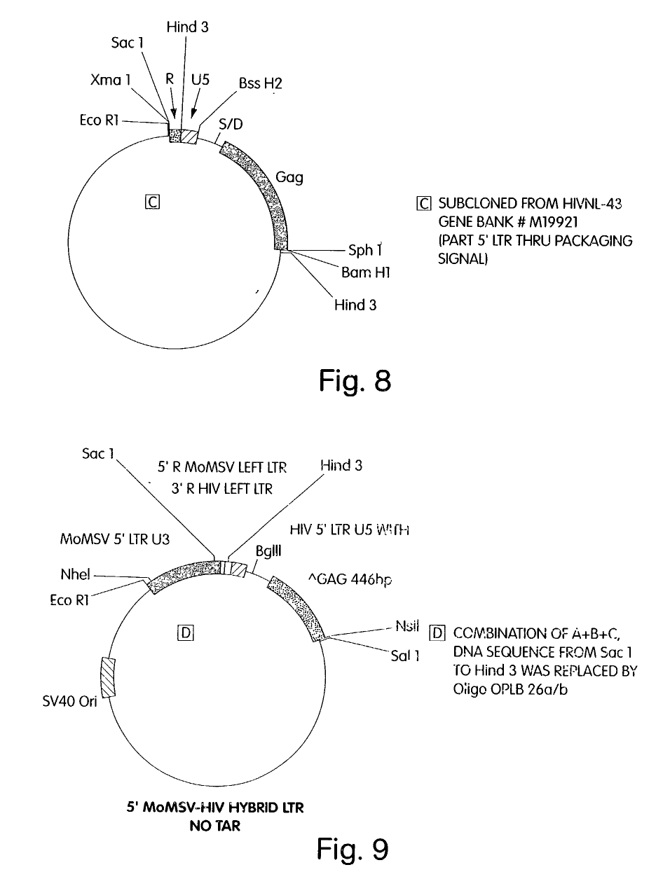

Plasmid C (FIG. 8) was subcloned from HIVNL-43 (Genbank Accession No. M19921) and contained the HIV 5′ LTR from the R region through the U5 region, and through the packaging signal at the start of GAG sequence.

Plasmid D

Plasmid D (FIG. 9) was formed by combining plasmids A, B, and C to form the complete 5′ hybrid LTR followed by the start of the HIV GAG sequence. The plasmid contained (within a Puc 19 backbone): the MoMSV U3 region (SEQ ID NO: 12), the left portion of the MoMSV R region (SEQ ID NO: 10), the right portion of the HIV R region without TAR (SEQ ID NO: 9), the HIV U5 region with att site (SEQ ID NO: 13), and the start of the HIV GAG sequence. The Sac 1 and Hind 3 sites of Plasmid D were used to insert annealed oligo (OPLB 26a/b) (SEQ ID NO: 4) containing a small piece of MoMSV U3 region and the hybrid R junction of MoMSV and HIV with the complete removal of the TAR region.

Plasmid E

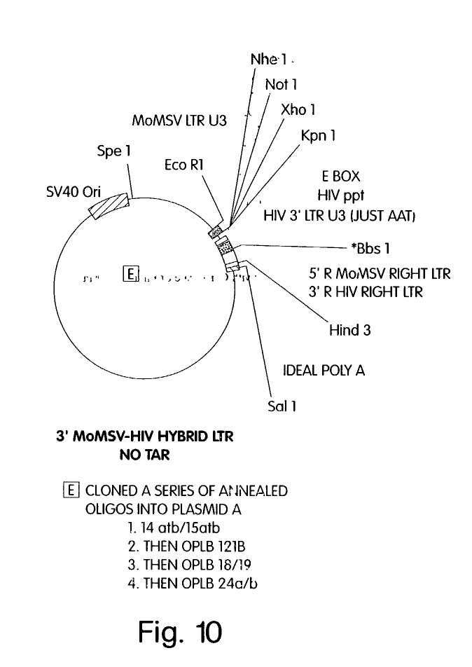

Plasmid E (FIG. 10) contained the complete 3′ hybrid LTR. The plasmid was constructed by cloning a series of annealed oligos into Plasmid A. Annealed oligo OPLB 14a+b/15a+b (SEQ ID NO: 5) was ligated into the Xho 1-Hind 3 sites in plasmid A resulting in the initial 3′ LTR containing HIV ppt, deleted HIV U3 with just the att site remaining, and part of MoMSV-HIV R region. Next, annealed oligo OPLB 12/13 (SEQ ID NO: 6) was ligated into the Hind 3-Sal 1 restriction sites resulting in the addition of the rest of the HIV R region and rabbit B globin poly A. Annealed oligo OPLB 18/19 (SEQ ID NO: 7) was then ligated into the Xho 1-Sac 1 restrictions sites which added the E-Box in front of the HIV ppt region. Finally, annealed oligo OPLB 24a/b (SEQ ID NO: 8) was ligated into restriction sites Bbs°1-Hind 3 to recreate the MoMSV-HIV R junction with the the entire TAR region being removed.

### Example 2

### Cell Culture, Transfections, Infections and Selection

The hybrid MoMSV-HIV vector constructed as described in Example 1 was transfected into 293T cells along with a gagpol plasmid vector containing HIV gagpol and Vif (no other accessory proteins), a rev expression vector, and VSVg (envelope) expression vector.

Transfections were performed using the calcium phosphate method (5′3′,Inc. and Gibco, Inc. kits). The following amounts of DNA were used for transfecting a 10 cm plate of 293T cells; 8 μg of hybrid vector, 12 μg of gagpol vector, 1 μg of rev vector, and 1 μg VSVg vector. When standard lentiviral vectors were used as a positive control, an additional 1 μg of a TAT expression vector DNA was added. When standard murine retroviral vectors were used as a negative control, 10 μg of hybrid vector, 8 μg gagpol vector, and 1 μg of VSVg vector were used per 10 cm dish.

Transfections were also performed using the Fugene (Roche) method which uses less DNA. Using this method, the following amounts of DNA were used for transfecting a 10 cm plate of 293T cells; 3.2 μg of hybrid vector, 4 μg of gagpol vector, 0.4 μg of rev vector, and 0.4 μg of VSVg vector. When standard lentiviral vectors were used as a positive control, an additional 0.4 μg of a TAT expression vector DNA was added. When standard murine retroviral vectors were used as a negative control, 4 μg of hybrid vector, 3.2 μg of gagpol vector, and 0.4 μg of VSVg vector were used per 10 cm dish.

Viral supernatants were collected 48 hours after transfection and filtered through a 0.45μ filter. Viral supernatants were then used to infect different cell types, such as 3T3 and Hela cells, and viral titers were measured as shown in Tables 1-3 below.

Table 1

Table 1 shows titers obtained in 3T3 cells for several MoMSV-HIV hybrid vectors (referred to as PLB vectors) containing various additional genetic elements as listed in the table. All of the vectors were “hybrid” in that they contained the same hybrid left (5′) and right (3′) LTRs as constructed and described in Example 1. The cells infected with Neomycin expressing vectors were split 1:10 into G418 (Gibco) and selected for ten days. The resulting colonies were counted and multiplied by the dilution factor to obtain the viral titers.

TABLE 1
MoMSV-HIV Hybrid Vector G418 Titers

| | | | | | | | Mean of |
|---|---|---|---|---|---|---|---|
| Vector | Expression Vector | TAT | Exp 1 | Exp 2 | Exp 3 | Exp 4 | Exp 1-4 |
| PLB 41 | PGK-Bgal-Neo | No | 6.95 | 17.2 | 10.7 | 21.1 | 14.0 |
| PLB 60 | Cppt PGK-Bgal-Neo | No | 12.6 | 63.9 | 13.1 | 12.7 | 25.6 |
| PLB 62 | PGK-Bgal-Neo-WPRE | No | 25.8 | 46.1 | 13.4 | 12.7 | 24.5 |
| PLB 65 | Cppt WPRE PGK-Bgal-Neo | No | 53.4 | 67.9 | 22.7 | 26.8 | 42.7 |
| PLB 67 | Cppt EF1-Bgal-Neo | No | 560 | 20.1 | 6.10 | — | 10.6 |
| PLB 71 | CMV-LTR PGK-Bgal-Neo | No | 1.55 | 11.5 | — | — | 6.5 |
| PLB 73 | CMV-LTR cppt PGK-Bgal- | No | 4.80 | 10.8 | — | — | 7.8 |
| | Neo | | | | | | |
| PLB 78 | Cppt EF1-Bgal-Neo WPRE | No | 16.0 | — | — | — | 16.0 |
| HPV 289 | Cppt PGK-Bgal-Neo | Yes | 382 | 424 | 132 | 89.0 | 215 |
| HPV 337 | Cppt EF1-Bgal-Neo | Yes | 290 | 570 | 141 | — | 355 |

Titiers are 10 to the 4^{th}
PLB vectors were the HIV-MoMSV Hybrid Vectors
HPV vectors were lentiviral HIV Vectors

Table 2

Viral titers were determined for 3T3 cells infected with GFP (green fluorescent protein) expressing vectors by FACS (Fluorescence activated cell sorter) analysis, the percentage of GFP positive cells were multiplied by the number of cells plated and the dilution factor. The results are shown below in Table 2.

TABLE 2
MoMSV-HIV Hybrid Vector GFP titers

| | | | | | | | Mean of |
|---|---|---|---|---|---|---|---|
| Vector | Expression Vector | TAT | Exp 1 | Exp 2 | Exp 3 | Exp 4 | Exp 1-4 |
| PLB 61 | PGK-GFP | No | 2.10 | 5.17 | — | 1.70 | 2.99 |
| PLB 63 | Cppt PGK-GFP | No | 3.18 | 1.79 | 3.55 | 2.49 | 2.75 |
| PLB 64 | Cppt EF1-GFP | No | 2.87 | 1.96 | 4.00 | 2.56 | 2.85 |
| PLB 76 | Cppt PGK-GFP WPRE | No | — | — | — | 3.02 | |
| PLB 77 | Cppt-EF1-GFP WPRE | No | — | — | — | 2.99 | |
| HPV 306 | Cppt PGK-GFP | Yes | 14.0 | 10.9 | 19.0 | 15.7 | 14.9 |
| HPV 335 | Cpt EF1-GFP1 | Yes | 11.0 | 18.9 | 14.2 | 14.5 | 14.6 |

Titiers are 10 to the 5^{th}
PLB - MoMSV-HIV Hybrid Vectors
HPV - Lentiviral Vectors

Table 3

The MoMSV-HIV hybrid vectors were also tested and shown to infect non-dividing cells, particularly Aphidicolin arrested Hela cells, as shown below in Table 3. Two controls were used: (1) standard murine retroviral vector (SM 10) which is not capable of infecting non-dividing cells and (2) standard lentiviral vectors (HPV 306 and HPV 335) which are capable of infecting non-dividing cells.

TABLE 3
MoMSV-HIV Hybrid Vector Results in Non-dividing Hela Cells

| Vector | Expression Vector | TAT | Hela | Hela ND |
|---|---|---|---|---|
| PLB 61 | PGK-GFP | No | 95.63% | 71.76% |
| PLB 63 | Cppt PGK-GFP | No | 99.47% | 77.64% |
| PLB 64 | Cppt EF1-GFP | No | 100% | 97.88% |
| SM 10 | IRES-GFP (Negative Control) | No | 98.81% | 0.15% |
| HPV 306 | Cppt PUK-GFP | Yes | 94.28% | 45.39% |
| HPV 335 | Cppt EFI-GFP | Yes | 99.80% | 57.42% |

% of GFP positive cells
PLB — MoMS V-I-IIV Hybrid Vectors
SM — Murine Retrovirus Vector
HPV — Lentiviral Vectors

In addition to the above studies, the hybrid lentiviral vectors of the present invention were also tested in a split HIV gagpol packaging system, in which the HIV GAG-Protease (GAG-PR) sequence is on one plasmid and the HIV Reverse Transcriptase-integrase (RT-IN) sequence is fused to the VPR on a separate plasmid as described in U.S. Ser. No. 09/311,684, the entire contents of which are incorporated by reference herein. In these studies, viral titers of 10⁵ were obtained. The protocol used for testing viral titers using the split gagpol system was the same as the protocol used for testing viral titers using the standard gagpol expression plasmid described above.

### Example 3

### In vivo Expression of Hybrid SIN Lentiviral Vectors

A. In vivo expression of hybrid SIN lentiviral vectors expressing GFP

Murine bone marrow cells from donor mice were transduced with concentrated and unconcentrated viral stocks containing hybrid self-inactivating (SIN) lentiviral vectors expressing green fluorescent protein (GFP). These cells were then used to reconstitute recipient mice. Two months later, blood cells from recipient mice were collected. The red blood cells were lysed using ammonium chloride and the white blood cells stained with a flourescently labeled antibody that recognizes an antigen called CD45.1. CD45.1 is present on blood cells of the recipient mice but is not expressed on any cell derived from the bone marrow cells of the donor mice. Use The proportion of blood cells derived from the donor and recipient mice are thus easily quantified through the use of the CD45.1 antibody.

FACS analysis of the blood cells collected from the recipient mice was performed to determine the number of cells expressing the GFP protein. The cells expressing GFP correspond to the number of cells expressing the transduced hybrid SIN lentiviral vectors of the invention.

The results showed that 55% to 61.46% of blood cells of donor origin in the recipient mouse contained the transduced hybrid SIN lentiviral vectors of the invention. These ranges were obtained when donor bone marrow cells exposed to high titer concentrated viral stocks were used to reconstitute recipient mice. When donor bone marrow cells exposed to unconcentrated virus stock were used, 4.36% of blood cells of donor origin contained the transduced hybrid SIN lentiviral vectors of the invention.

B. In vivo expression of hybrid SIN lentiviral vectors expressing β-globin

Murine bone marrow cells from donor mice were transduced with concentrated viral stocks containing hybrid self-inactivating (SIN) lentiviral vectors containing the gene encoding human β-globin. These cells were then used to reconstitute recipient mice. Two months later, blood cells from recipient mice were collected. The red blood cells were fixed and treated with a chemical that creates holes in the cell membrane. The cells were then exposed to a flourescently labeled antibody that specifically recognizes the human β-globin gene. This antibody can not permeate the cell membrane and bind to the β-globin protein unless holes in the cell membrane are chemically created.

FACS analysis of the blood cells collected from the recipient mice was performed to determine the number of cells expressing the β-globin protein. The cells expressing β-globin correspond to the number of cells expressing the transduced hybrid SIN lentiviral vectors of the invention.

The results showed that 15.52% to 22.28% of blood cells of donor origin in the recipient mouse contained the transduced hybrid SIN lentiviral vectors of the invention.

### CONCLUSION

The results of the in vitro and in vivo studies described above demonstrate that the hybrid self-inactivating (SIN) lentiviral vectors of the present invention generate high viral titers in the range known to be useful for human gene therapy. Specifically, titers in the range of 10⁴ to 10⁵ can be obtained (depending on the size of the insert, smaller inserts achieving greater titers) prior to concentration, and in the range of 10⁷ to 10⁹ after concentration. Moreover, there was no indication of replication-competent virus. The results also demonstrate that the the hybrid lentiviral vectors of the present invention are efficient at transducing non-dividing cells, further adding to their usefulness in gene therapy.

What is claimed is:

1. A retroviral LTR comprising a lentiviral R region which lacks all or a portion of the TAR sequence.

2. The retroviral LTR of claim 1, wherein the TAR sequence or portion thereof is replaced by a comparable portion of the R region from a non-lentiviral retrovirus, thereby forming a hybrid R region.

3. The retroviral LTR of claim 1, wherein the lentivirus is selected from the group consisting of HIV, EIV, SIV and FIV.

4. The retroviral LTR of claim 1, wherein the lentivirus is HIV-1 or HIV-2.

5. The retroviral LTR of claim 2, wherein the non-lentiviral retrovirus is selected from the group consisting of MoMSV, MoMLV, MLV, Friend, MSCV, RSV and spumavirus.

6. The retroviral LTR of claim 2, wherein the lentivirus is HIV and the non-lentiviral retrovirus is MoMSV.

7. The retroviral LTR of claim 6, wherein the hybrid R region comprises a portion of the HIV R region comprising the nucleotide sequence shown in SEQ ID NO: 10 and a portion of the MoMSV R region comprising the nucleotide sequence shown in SEQ ID NO: 9.

8. The retroviral LTR of claim 6, wherein the hybrid R region comprises the nucleotide sequence shown in SEQ ID NO: 11.

9. A retroviral LTR comprising a hybrid R region, wherein the hybrid R region comprises: (1) a portion of a lentiviral R region lacking the TAR sequence; and (b) a comparable portion of an R region from a non-lentiviral retrovirus.

10. The retroviral LTR of claim 9, wherein the portion of the lentiviral R region is from a lentivirus selected from the group consisting of HIV, EIV, SIV and FIV.

11. The retroviral LTR of claim 9, wherein the portion of the R region from the non-lentiviral retrovirus is from a retrovirus selected from the group consisting of MoMSV, MoMLV, MLV, Friend, MSCV, RSV and spumavirus.

12. The retroviral LTR of claim 9, wherein the portion of the lentiviral R region is from HIV and the portion of the R region from the non-lentiviral retrovirus is from MoMSV.

13. The retroviral LTR of claim 12, wherein the hybrid R region comprises a portion of the HIV R region comprising the nucleotide sequence shown in SEQ ID NO: 10 and a portion of the MoMSV R region comprising the nucleotide sequence shown in SEQ ID NO: 9.

14. The retroviral LTR of claim 12, wherein the hybrid R region comprises the nucleotide sequence shown in SEQ ID NO: 11.

15. The retroviral LTR of claim 9, wherein the LTR is a left (5′) LTR and further comprises a promoter sequence upstream from the hybrid R region.

16. The retroviral LTR of claim 15, wherein the promoter sequence comprises the U3 region of a non-lentiviral retrovirus.

17. The retroviral LTR of claim 15, wherein the promoter sequence comprises the MoMSV U3 region.

18. The retroviral LTR of claim 15 further comprising a lentiviral U5 region downstream from the hybrid R region.

19. The retroviral LTR of claim 18, wherein the U5 region is the HIV U5 region including the HIV att sequence.

20. The retroviral LTR of claim 9, wherein the LTR is a right (3′) LTR and further comprises a portion of a lentiviral U3 region upstream from the hybrid R region, wherein the portion includes the lentiviral U3 att sequence but lacks any promoter activity.

21. The retroviral LTR of claim 20, wherein the portion comprises the 3′ end of the lentiviral U3 region up to and including the lentiviral U3 att sequence.

22. The retroviral LTR of claim 20, wherein the portion of the U3 region is from HIV.

23. The retroviral LTR of claim 20, wherein the portion of the U3 region comprises the nucleotide sequence shown in SEQ ID NO: 15.

24. The retroviral LTR of claim 20 further comprising a polyadenylation sequence downstream from the hybrid R region.

25. The retroviral LTR of claim 24, wherein the polyadenylation sequence comprises the rabbit β-globin gene polyadenylation sequence.

26. The retroviral LTR of claim 24, wherein the polyadenylation sequence comprises the nucleotide sequence shown in SEQ ID NO: 16.

27. A retroviral LTR comprising a hybrid R region, wherein the hybrid R region comprises: (1) a portion of the HIV R region lacking the TAR sequence; and (b) a portion of the MoMSV R region.

28. The retroviral LTR of claim 20, wherein the portion of the HIV R region comprises the nucleotide sequence shown in SEQ ID NO: 10.

29. The retroviral LTR of claim 20, wherein the portion of the MoMSV R region comprises the nucleotide sequence shown in SEQ ID NO: 9.

30. The retroviral LTR of claim 20, wherein the hybrid R region comprises the nucleotide sequence shown in SEQ ID NO: 11.

31. A left (5′) retroviral LTR comprising three regions, U3, R and U5, wherein:
the U3 region comprises a promoter sequence;
the R region comprises a combination of (a) ) a portion of an R region from a non-lentiviral retrovirus, and (b) a portion of an R region from a lentivirus which does not include the TAR sequence; and
the U5 region comprises a lentiviral U5 region.

32. The left (5′) retroviral LTR of claim 31, wherein the U3 region comprises the U3 region from a non-lentiviral retrovirus.

33. The left (5′) retroviral LTR of claim 31, wherein the U3 region comprises the MoMSV U3 region.

34. The left (5′) retroviral LTR of claim 31, wherein the R region comprises a portion of the MoMSV R region, and a portion of the HIV R region lacking the TAR sequence..

35. The left (5′) retroviral LTR of claim 31, wherein the U5 region comprises the HIV U5 region.

36. A left (5′) retroviral LTR comprising three regions referred to, from left to right, as U3-R-U5, wherein:
the U3 region comprises the MoMSV U3 region;
the R region comprises the left half of the MoMSV R region and the right half of the HIV R region lacking the HIV TAR sequence but including the HIV polyadenylation sequence; and
the U5 region comprises the HIV U5 region including the HIV att sequence.

37. The left (5′) retroviral LTR of claim 36, wherein the U3 region comprises the nucleotide sequence shown in SEQ ID NO: 12.

38. The left (5′) retroviral LTR of claim 36, wherein the R region comprises the nucleotide sequence shown in SEQ ID NO: 11.

39. The left (5′) retroviral LTR of claim 36, wherein the U5 region comprises the a nucleotide sequence shown in SEQ ID NO: 13.

40. A left (5′) retroviral LTR comprising the nucleotide sequence shown in SEQ ID NO: 14.

41. A right (3′) retroviral LTR comprising three regions, U3, R and P, wherein:
the U3 region comprises the att sequence from a lentiviral U3 region but lacks any sequences from the U3 region having promoter activity;
the R region comprises a combination of (a) a portion of an R region from a non-lentiviral retrovirus, and (b) a portion of an R region from a lentivirus which does not include the TAR sequence; and
the P region comprises a polyadenylation sequence.

42. The right (3′) retroviral LTR of claim 41, wherein the U3 region comprises the HIV att sequence.

43. The right (3′) retroviral LTR of claim 41, wherein the U3 region comprises the nucleotide sequence shown in SEQ ID NO: 15.

44. The right (3′) retroviral LTR of claim 41, wherein the R region comprises a portion of the MoMSV R region, and a portion of the HIV R region lacking the TAR sequence.

45. The right (3′) retroviral LTR of claim 41, wherein the R region comprises the nucleotide sequence shown in SEQ ID NO: 11.

46. The right (3′) retroviral LTR of claim 41, wherein the P region comprises the rabbit β-globin gene polyadenylation sequence.

47. The right (3′) retroviral LTR of claim 41, wherein the P region comprises the nucleotide sequence shown in SEQ ID NO: 16.

48. A right (3′) retroviral LTR comprising three regions referred to, from left to right, as U3-R-P, wherein:
the U3 region comprises a portion of the HIV U3 region containing the HIV att sequence but not containing any sequences from the HIV U3 region having promoter activity;
the R region comprises the left half of the MoMSV R region and the right half of the HIV R region lacking the HIV TAR sequence but including the HIV polyadenylation sequence; and
the P region comprises a polyadenylation sequence.

49. The right (3′) retroviral LTR of claim 48, wherein the U3 region comprises the nucleotide sequence shown in SEQ ID NO: 15.

50. The right (3′) retroviral LTR of claim 48, wherein the R region comprises the nucleotide sequence shown in SEQ ID NO: 11.

51. The right (3′) retroviral LTR of claim 48, wherein the P region comprises the a nucleotide sequence shown in SEQ ID NO: 16.

52. The right (3′) retroviral LTR of claim 48 further comprising the HIV polypurine track (ppt) sequence upstream from the U3 region.

53. A right (3′) retroviral LTR comprising the nucleotide sequence shown in SEQ ID NO: 17.

54. A hybrid retroviral LTR comprising all or a portion of the R region from a non-lentiviral retrovirus and all or a portion of the U3 region or the U5 region from a lentivirus.

55. The hybrid retroviral LTR of claim 54, wherein the LTR is a left (5′) LTR and comprises all or a portion of the U3 region of a non-lentiviral retrovirus, all or a portion of the R region of a non-lentiviral retrovirus, and all or a portion of the U5 region from a lentivirus.

56. The hybrid retroviral LTR of claim 54, wherein the LTR is a right (5′) LTR and comprises all or a portion of the U3 region of a lentivirus and all or a portion of the R region of a non-lentiviral retrovirus.

57. The hybrid retroviral LTR of any one of claims 54, 55 or 56, wherein the lentivirus is HIV and the non-lentiviral retrovirus is MoMSV.

58. An expression vector comprising the retroviral LTR of any one of claims 1, 9, 20, 31, 36, 41, 48 or 54.

59. The expression vector of claim 58, wherein the expression vector is a SIN vector.

60. An expression vector comprising
(1) a left (5′) retroviral LTR comprising three regions, U3, R and U5, wherein:
the U3 region comprises a promoter sequence;
the R region comprises a combination of (a) ) a portion of an R region from a non-lentiviral retrovirus, and (b) a portion of an R region from a lentivirus which does not include the TAR sequence; and
the U5 region comprises a lentiviral U5 region; and further comprising
(2) a right (3′) retroviral LTR comprising three regions, U3, R and P, wherein:
the U3 region comprises the att sequence from a lentiviral U3 region but lacks any sequences from the U3 region having promoter activity;
the R region comprises a combination of (a) a portion of an R region from a non-lentiviral retrovirus, and (b) a portion of an R region from a lentivirus which does not include the TAR sequence; and
the P region comprises a polyadenylation sequence.

61. The expression vector of claim 60, wherein the expression vector is a SIN vector.

62. The expression vector of claim 60 further comprising a polypurine tract upstream from the U3 region of the right (3′) LTR.

63. The expression vector of claim 60 further comprising an RNA export element.

64. The expression vector of claim 60, wherein the RNA export element comprises the HIV Rev responsive element (RRE).

65. The expression vector of claim 60 further comprising an exogenous gene.

66. The expression vector of claim 60, wherein the gene is a marker gene.

67. The expression vector of claim 66, wherein the marker gene comprises the green fluorescence protein (GFP) gene.

68. The expression vector of claim 60, wherein the gene is a therapeutic gene.

69. The expression vector of claim 68, wherein the gene encodes a protein which promotes angiogenesis.

70. An expression vector comprising
(1) a left (5′) retroviral LTR comprising three regions referred to, from left to right, as U3-R-U5, wherein:
the U3 region comprises the MoMSV U3 region;
the R region comprises the left half of the MoMSV R region and the right half of the HIV R region lacking the HIV TAR sequence but including the HIV polyadenylation sequence; and
the U5 region comprises the HIV U5 region including the HIV att sequence; and further comprising
(2) a right (3′) retroviral LTR comprising three regions referred to, from left to right, as U3-R-P, wherein:
the U3 region comprises a portion of the HIV U3 region containing the HIV att sequence but not containing any sequences from the HIV U3 region having promoter activity;
the R region comprises the left half of the MoMSV R region and the right half of the HIV R region lacking the HIV TAR sequence but including the HIV polyadenylation sequence; and
the P region comprises a polyadenylation sequence.

71. The expression vector of claims 70, wherein the expression vector is a SIN vector.

72. The expression vector of claim 70 further comprising a polypurine tract upstream from the U3 region of the right (3′) LTR.

73. The expression vector of claim 70 further comprising an RNA export element.

74. The expression vector of claim 70, wherein the RNA export element comprises the HIV Rev responsive element (RRE).

75. The expression vector of claim 70 further comprising an exogenous gene.

76. The expression vector of claim 70, wherein the gene is a marker gene.

77. The expression vector of claim 76, wherein the marker gene comprises the green fluorescence protein (GFP) gene.

78. The expression vector of claim 70, wherein the gene is a therapeutic gene.

79. The expression vector of claim 78, wherein the gene encodes a protein which promotes angiogenesis.

80. The expression vector of any one of claims 65-69 or 75-79 further comprising a promoter sequence upstream from the gene.

81. The expression vector of claim 80, wherein the promoter is selected from the group consisting of the PGK, EF1 alpha and CMV promoter.

82. The expression vector of claim 80, wherein the promoter is the natural promoter associated with the gene.

83. The expression vector of claim 80 further comprising an internal ribosome entry site (IRES).

84. The expression vector of claim 80 further comprising all or a portion of a lentiviral GAG sequence.

85. The expression vector of claim 80, wherein the gene is selected from the group consisting of genes encoding soluble Interleukin-1α Receptor Type I, Soluble Interleukin-1α Receptor Type II, Interleukin-1α Receptor Antagonist Protein (IRAP), Insulin-Like Growth Factor (IGF), Tissue Inhibitors of Matrix Metallo-Proteinases (TIMP)-1,-2,-3,-4, Bone Morphogenic Protein (BMP)-2 and -7, Indian Hedgehog, Sox-9, Interleukin-4, Transforming Growth Factor (TGF)-β, Superficial Zone Protein, Cartilage Growth and Differentiation Factors (CGDF), Bcl-2, Soluble Tumor Necrosis Factor (TNF)-α Receptor, Fibronectin and/or Fibronectin Fragments, Leukemia Inhibitory Factor (LIF), LIF binding protein (LBP), Interleukin-4, Interleukin-10, Interleukin-11, Interleukin-13, Hyaluronan Synthase, soluble TNF-α receptors 55 and 75, Insulin Growth Factor (IGF)-1, activators of plasminogen, urokinase plasminogen activator (uPA), parathyroid hormone-related protein (PTHrP), and platelet derived growth factor (PDGF)-AA -AB or -BB.

86. Use of the expression vector of any one of claims 60-79 in gene therapy.

87. A virion for use in gene therapy comprising the expression vector of any one of claims 60-79.

88. A method of delivering a gene to a cell comprising contacting the cell with the virion of claim 87.

89. The method of claim 88, wherein the cell is selected from the group comprising an autologous cell, a bone marrow cell, a mesenchymal stem cell obtained from adipose tissue, a synovial fibroblast or a chondrocyte, a non-autologous cell (i.e., is allogeneic or xenogenic) and a cell from a cell line or from primary cells derived from a human or animal source.

90. The method of claim 88, wherein the virion is contacted with the cell in vivo.

91. The method of claim 88, wherein the virion is contacted with the cell ex vivo.

92. A method of producing a lentivirus suitable for use in gene therapy comprising transfecting the expression vector of any one of claims 60-79 into a packaging cell line containing the necessary envelop (env) and polymerase (pol) gene sequences to produce the lentivirus.

93. The method of claim 92, wherein the envelope (env) gene is from a non-lentiviral virus so that the lentivirus is pseudotyped.


SEQUENCE LISTING (17 sequences)

SEQ ID NO: 1 - DNA, 651 bp, Human immunodeficiency virus type 1
gaattctttgaaagaccccacccgtaggtggcaagctagcttaagtaacgccactttgcaaggcatggaaaaatacataactgagaataggaaagttcagatcaaggtcaggaacaaagaaacagctgaataccaaacaggatatctgtggtaagcggttcctgccccggctcagggccaagaacagatgagacagctgagtgatgggccaaacaggatatctgtggtaagcagttcctgccccggctcggggccaagaacagatggtccccagatgcggtccagccctcagcagtttctagtgaatcatcagatgtttccagggtgccccaaggacctgaaaatgaccctgtaccttatttgaactaaccaatcagttcgcttctcgcttctgttcgcgcgcttccgctctccgagctcaataaaagagcccacaacccctcactcggcgcgccagtcttccgatagactgcgtcgcactgcttaagcctcaataaagcttgccttgagtgcttcaaagtagtgtgtgcccgtctgttgtgtgactctggtaactagagatccctcagacccttttagtcagtgtggaaaatctctagcagtggcgcccgaacagggacttgaaagcgaaagtaaagccagaggagatct

SEQ ID NO: 2 - DNA, 210 bp, Human immunodeficiency virus type 1
ctcgagggtacctttaagaccaatgacttacaaggcagctgtagatcttagccactttttaaaagaaaaggggggactggaagggctaattcactcccaaagaagacaagatatgcgccagtcttccgatagactgcgtcgcactgcttaagcctcaataaagcttgccttgagtgcttcaatgtgtgtgttggttttttgtgtgtcgac

SEQ ID NO: 3 - DNA, 82 bp, Human immunodeficiency virus type 1
gaattctttgaaagaccccacccgtaggtggcaagctagcggccgctcgagaatcgataagctttctagagtcgactagctt

SEQ ID NO: 4 - DNA, 88 bp, Human immunodeficiency virus type 1
gagctcaataaaagagcccacaacccctcactcggcgcgccagtcttccgatagactgcgtcgcactgcttaagcctcaataaagctt

SEQ ID NO: 5 - DNA, 148 bp, Human immunodeficiency virus type 1
ctcgagtttttaaaagaaaaggggggactggaagggctaattcactcccaaagaagacaagatatgcgccagtcttccgatagactgcgtcgcccggggagctctctggctaactagggaacccactgcttaagcctcaataaagctt

SEQ ID NO: 6 - DNA, 50 bp, Human immunodeficiency virus type 1
aagcttgccttgagtgcttcaatgtgtgtgttggttttttgtgtgtcgac

SEQ ID NO: 7 - DNA, 153 bp, Human immunodeficiency virus type 1
ctcgagggtacctttaagaccaatgacttacaaggcagctgtagatcttagccactttttaaaagaaaaggggggactggaagggctaattcactcccaaagaagacaagatatgcgccagtcttccgatagactgcgtcgcccggggagctc

SEQ ID NO: 8 - DNA, 65 bp, Human immunodeficiency virus type 1
gaagacaagatatgcgccagtcttccgatagactgcgtcgcactgcttaagcctcaataaagctt

SEQ ID NO: 9 - DNA, 28 bp, Human immunodeficiency virus type 1
gcgccagtcttccgatagactgcgtcgc

SEQ ID NO: 10 - DNA, 39 bp, Human immunodeficiency virus type 1
actgcttaagcctcaataaagcttgccttgagtgcttca

SEQ ID NO: 11 - DNA, 67 bp, Human immunodeficiency virus type 1
gcgccagtcttccgatagactgcgtcgcactgcttaagcctcaataaagcttgccttgagtgcttca

SEQ ID NO: 12 - DNA, 450 bp, Human immunodeficiency virus type 1
gaattctttgaaagaccccacccgtaggtggcaagctagcttaagtaacgccactttgcaaggcatggaaaaatacataactgagaataggaaagttcagatcaaggtcaggaacaaagaaacagctgaataccaaacaggatatctgtggtaagcggttcctgccccggctcagggccaagaacagatgagacagctgagtgatgggccaaacaggatatctgtggtaagcagttcctgccccggctcggggccaagaacagatggtccccagatgcggtccagccctcagcagtttctagtgaatcatcagatgtttccagggtgccccaaggacctgaaaatgaccctgtaccttatttgaactaaccaatcagttcgcttctcgcttctgttcgcgcgcttccgctctccgagctcaataaaagagcccacaacccctcactcggc

SEQ ID NO: 13 - DNA, 84 bp, Human immunodeficiency virus type 1
aagtagtgtgtgcccgtctgttgtgtgactctggtaactagagatccctcagacccttttagtcagtgtggaaaatctctagca

SEQ ID NO: 14 - DNA, 601 bp, Human immunodeficiency virus type 1
gaattctttgaaagaccccacccgtaggtggcaagctagcttaagtaacgccactttgcaaggcatggaaaaatacataactgagaataggaaagttcagatcaaggtcaggaacaaagaaacagctgaataccaaacaggatatctgtggtaagcggttcctgccccggctcagggccaagaacagatgagacagctgagtgatgggccaaacaggatatctgtggtaagcagttcctgccccggctcggggccaagaacagatggtccccagatgcggtccagccctcagcagtttctagtgaatcatcagatgtttccagggtgccccaaggacctgaaaatgaccctgtaccttatttgaactaaccaatcagttcgcttctcgcttctgttcgcgcgcttccgctctccgagctcaataaaagagcccacaacccctcactcggcgcgccagtcttccgatagactgcgtcgcactgcttaagcctcaataaagcttgccttgagtgcttcaaagtagtgtgtgcccgtctgttgtgtgactctggtaactagagatccctcagacccttttagtcagtgtggaaaatctctagca

SEQ ID NO: 15 - DNA, 38 bp, Human immunodeficiency virus type 1
ctggaagggctaattcactcccaaagaagacaagatat

SEQ ID NO: 16 - DNA, 24 bp, Human immunodeficiency virus type 1
atgtgtgtgttggttttttgtgtg

SEQ ID NO: 17 - DNA, 129 bp, Human immunodeficiency virus type 1
ctggaagggctaattcactcccaaagaagacaagatatgcgccagtcttccgatagactgcgtcgcactgcttaagcctcaataaagcttgccttgagtgcttcaatgtgtgtgttggttttttgtgtg